(19) United States
(12) Patent Application Publication
(10) Pub. No.: US 2026/0194539 A1
(43) Pub. Date: Jul. 9, 2026

(54) METHOD FOR DIAGNOSING EARLY AND CLASSIFYING STAGES OF ALZHEIMER'S DISEASE AND DETERMINING AMYLOID BETA ACCUMULATION IN BRAIN BY USING TAU PROTEIN-DERIVED PHOSPHORYLATED PEPTIDE

(71) Applicants: GWANGJU INSTITUTE OF SCIENCE AND TECHNOLOGY, Gwangju (KR); SEOUL NATIONAL UNIVERSITY R&DB FOUNDATION, Seoul (KR); INDUSTRY-ACADEMIC COOPERATION FOUNDATION, CHOSUN UNIVERSITY, Gwangju (KR)

(72) Inventors: ZEE-YONG PARK, Gwangju (KR); SE HWAN JANG, Gwangju (KR); YOU RIM KIM, Gwangju (KR); DAEHEE HWANG, Daegu (KR); KUNHO LEE, Gwangju (KR)

(21) Appl. No.: 18/867,995

(22) PCT Filed: Jun. 30, 2023
(86) PCT No.: PCT/KR2023/009231

§ 371 Date: Nov. 21, 2024

(30) Foreign Application Priority Data
Jun. 30, 2022 (KR) 10-2022-0080863

(51) Int. Cl. G01N 33/68; C12Q 1/34; G01N 30/02; G01N 30/72; G01N 30/86; G01N 30/88

(52) U.S. Cl. CPC G01N 33/6896; C12Q 1/34; G01N 30/7233; G01N 30/86; G01N 33/6848; G01N 2030/027; G01N 2030/8831; G01N 2333/4709; G01N 2333/976; G01N 2440/14; G01N 2800/2821

(57) ABSTRACT

A method for early diagnosis or stage classification of Alzheimer's disease includes obtaining a tau protein-derived phosphorylated peptide from an isolated biological sample and quantifying the tau protein-derived phosphorylated peptide. Through the quantification of tau protein-derived phosphorylated peptides, it is possible to effectively diagnose Alzheimer's disease at an early stage or classify its stages, and determine the accumulation of amyloid beta in the brain.

### REFERENCE TO AN ELECTRONIC SEQUENCE LISTING

A sequence listing electronically submitted on Nov. 21, 2024 as a xml file named 20241121_LC0982427_TU_SEQ.XML, created on Nov. 21, 2024 and having a size of 27,752 bytes, is incorporated herein by reference in its entirety.

### BACKGROUND

### 1. Field of the Invention

The present invention relates to a method for early diagnosis of Alzheimer's disease using a tau protein-derived phosphorylated peptide, and more specifically, to a method for early diagnosis or stage classification of Alzheimer's disease by measuring the phosphorylation level of tau protein in a biological sample, and a method for determination of amyloid beta accumulation in the brain.

### 2. Background Art

Alzheimer's disease is one of the biggest causes of dementia, and interest is growing as the number of patients is increasing. However, until now, the exact cause has not been identified and no specific therapeutics have been found. At present, clinically used diagnosis of Alzheimer's disease involves observation of changes in specific brain tissues (hippocampus, cortex, etc.) using magnetic resonance imaging (MRI), single-photon emission computed tomography (SPECT) and positron emission tomography (PET), etc. A diagnosis of Alzheimer's disease is made by performing observations as described above and combining observations of changes in behavior and perception through questionnaires such as mini mental status examination (MMSE).

However, the above methods have limitations in that they are expensive and most methods allow diagnosis only after the disease progresses and symptoms appear. Currently, the only way to confirm the diagnosis is postmortem analysis of brain tissue through autopsy after the patient dies, which determines whether amyloid beta plaques are deposited in brain cells and neurofibrillary tangles formed by hyperphosphorylation of tau protein. As such, diagnosis is difficult and there is no treatment method for patients who have passed the stage of mild cognitive dysfunction and have reached dementia, therefore, a need to develop drugs targeting early-stage patients and the importance of selecting early-stage patients and precise early diagnosis are being highlighted. Alzheimer's disease has two stages: a “preclinical” stage, in which the patient's behavior appears normal but pathological changes occur in the brain; and a “mild cognitive impairment (MCI)” stage, in which mild cognitive degradation occurs at the stage before symptoms of the disease appear in most cases. Thus, if Alzheimer's disease can be diagnosed at this early stage, more effective treatment will be possible. Further, the determination of amyloid beta accumulation in the brain, which is the causative material of Alzheimer's dementia and has a significant correlation with the progression of the disease, is the basis for sub-classifying patients with Alzheimer's disease and non-Alzheimer's disease even in patients at different stages of dementia with the same extent of cognitive function, such that it may be used for customized treatment for each patient and for additional diagnosis in the future.

Currently, researches are being actively conducted on methods to early diagnose Alzheimer's disease and determine amyloid beta accumulation in the brain by measuring protein levels that are specifically increased or decreased in the blood or cerebrospinal fluid of Alzheimer's disease patients before appearance of symptoms. However, the cerebrospinal fluid is painful during the sample collection process, is not easy to collect, and has concerns about medical side effects. Further, it is particularly difficult to collect the cerebrospinal fluid from elderly patients with Alzheimer's disease, and it cannot be easily used as a general diagnostic method. On the other hand, blood is considered to be a suitable sample for developing an effective Alzheimer's disease biomarker based on the fact that it is easier to collect the blood than the cerebrospinal fluid and based on results of researches showing that 500 ml of cerebrospinal fluid is absorbed into the blood every day.

Accordingly, much research has been executed to use amyloid beta and tau proteins in the blood as early diagnosis markers. However, in the case of amyloid beta, completely conflicting results were obtained for each research group, and when analyzing the entire tau protein, the results showed that the concentration ranges of tau protein in the Alzheimer's disease patient group and the normal control overlapped greatly, such that its utility and specificity as an indicator for early diagnosis were very deteriorated. On the other hand, the hyperphosphorylation phenomenon of tau protein in the blood was found to be more specific to Alzheimer's disease than the existing tau protein overall and to have a higher correlation with amyloid beta lesions and tau lesions in the brain. Thus, research is underway to diagnose Alzheimer's disease using the hyperphosphorylation phenomenon of tau protein as an indicator in the blood. In particular, phosphorylated tau protein in the blood comprehensively reflects not only passive release due to death of neurons (neurodegeneration) from brain tissue, but also active release of tau protein depending on the phosphorylation site. Therefore, it is essential to directly measure a phosphorylation level of tau protein present in blood rather than brain tissues, and use results thereof as an indicator for early diagnosis, stage classification, and determination of amyloid beta accumulation in the brain. However, the current method for quantifying and diagnosing the phosphorylation of tau protein in the blood has been done only by digital enzyme-linked immunosorbent assay (ELISA), which is an ultra-sensitive immunoassay technology that digitally measures enzyme single molecule activity using a microwell array with a size of several tens of microns. This method has problems of immunoassay (low throughput, affinity and specificity of antibodies that vary depending on the sample and epitopes, and the need to develop antibodies for the epitopes to be quantified), as well as the analysis is performed at limited phosphorylation positions 181 and 217 of the tau protein. Therefore, an alternative method useable in actual clinical practice is required for early diagnosis of Alzheimer's disease, stage classification, and determination of amyloid beta accumulation in the brain.

### SUMMARY

As a result of research efforts to develop a test method for effective early diagnosis of Alzheimer's disease and determination of amyloid beta accumulation in the brain, the present inventors have simultaneously measured amounts of various phosphorylated peptides derived from tau proteins present in the blood using an efficient phosphorylated peptide enrichment process and liquid chromatography-mass spectrometry (LC-MS) device, thereby allowing early diagnosis and stage classification of Alzheimer's disease using Alzheimer's disease-specific tau protein phosphorylation sites and a difference in their phosphorylation levels, and have confirmed that amyloid beta accumulation in the brain could be determined, and therefore, the present invention has been completed on the basis of the above confirmation.

An object of the present invention is to provide a method for early diagnosis of Alzheimer's disease, stage classification, and determination of amyloid beta accumulation in the brain by obtaining and quantitatively analyzing tau protein-derived phosphorylated peptides in biological samples including blood and cerebrospinal fluid.

Another object of the present invention is to provide a biomarker and composition for early diagnosis of Alzheimer's disease, stage classification, and determination of amyloid beta accumulation in the brain.

Through the quantification of tau protein-derived phosphorylated peptides according to the present invention, it is possible to effectively diagnose Alzheimer's disease at an early stage or classify its stages, and determine the accumulation of amyloid beta in the brain.

### BRIEF DESCRIPTION OF THE DRAWINGS

FIG. 1 shows an overall schematic diagram for carrying out the method of the present invention.

FIG. 2 shows the specific steps for carrying out the method of the present invention.

FIG. 3 shows the quantification of phosphorylated peptides according to the present invention using numerical values.

FIG. 4 shows the quantification of phosphorylated peptides according to the present invention using a histogram.

FIG. 5 shows the quantification of phosphorylated peptides according to the present invention using a heatmap.

FIG. 6 shows a quantitative results of the phosphorylated peptide according to the present invention in terms of fold change compared to the normal control.

FIG. 7 shows a schematic diagram of phosphorylation sites that show a specific increase according to stages of Alzheimer's disease in the present invention or a specific change according to amyloid accumulation in the brain at the same stage.

FIG. 8 shows the results of quantifying phosphorylated peptides that can determine the preclinical Alzheimer's disease stage according to the present invention.

FIG. 9 shows the results of quantifying phosphorylated peptides that can determine Alzheimer's mild cognitive impairment according to the present invention.

FIG. 10 shows the results of quantifying phosphorylated peptides that can distinguish amyloid deposition at the same stage of MCI.

FIG. 11 shows the results of quantifying phosphorylated peptides that can distinguish amyloid deposition at the same stage of AD.

FIG. 12 shows a specific steps for selecting a patient group or a normal group corresponding to the Alzheimer's disease stage for a clinical trial through classification of Alzheimer's disease stage in the present invention.

### DETAILED DESCRIPTION

Hereinafter, the present invention will be described in detail. Meanwhile, each description and embodiment disclosed in the present invention may also be applied to each of other descriptions and embodiments. That is, all combinations of the various elements disclosed in the present invention fall within the scope of the present invention. Further, it is not to be considered that the scope of the present invention is limited by the specific description described below.

In one aspect for achieving the above objects, the present invention provides a method for early diagnosis or stage classification of Alzheimer's disease, including the steps of: (a) obtaining a tau protein-derived phosphorylated peptide from an isolated biological sample; and (b) quantifying the tau protein-derived phosphorylated peptide.

The above method may refer to a method of providing information for early diagnosis or stage classification of Alzheimer's disease.

In another aspect, the present invention provides a method for determination of amyloid beta accumulation in the brain, including the steps of: (a) obtaining a tau protein-derived phosphorylated peptide from an isolated biological sample; and (b) quantifying the tau protein-derived phosphorylated peptide.

The above method may refer to a method of providing information for determination of amyloid beta accumulation in the brain.

The above method may further include the step of (c) comparing the quantified tau protein-derived phosphorylated peptide with a control.

More specifically, the method of the present invention for early diagnosis of Alzheimer's disease, stage classification and determination of amyloid beta accumulation in the brain may include the steps of: converting proteins in the isolated biological sample into peptides using proteolytic enzymes; concentrating the tau protein-derived phosphorylated peptide by applying phosphorylated peptide enrichment process to the obtained peptides; quantifying the concentrated tau protein-derived phosphorylated peptide through mass spectrometry using the liquid chromatography-mass spectrometry (LC-MS) device; and comparing the quantified amount of phosphorylated peptide with a control.

The basic feature of the present invention is to develop a test method for effective early diagnosis of Alzheimer's disease, stage classification, and determination of amyloid beta accumulation in the brain, wherein, for patients who have not yet reached Alzheimer's disease in the preclinical Alzheimer's disease (preclinical AD) stage and/or Alzheimer's mild cognitive impairment (MCI) stage can be detected early, and accumulation of amyloid beta in the brain in the normal cognitive function group, mild cognitive impairment group or dementia group with the same level of cognitive function can be determined.

Conventionally, many studies with significant differences between actual Alzheimer's disease dementia patients and normal people have been conducted, but there was no significant difference between the preclinical Alzheimer's disease (preclinical AD) stage and/or Alzheimer's mild cognitive impairment (MCI) stage, which is the stage before the symptoms of the disease appear, and normal people, hence causing difficulties in early diagnosis. However, the present invention has developed a method for early diagnosis and determination of amyloid beta accumulation in the brain using blood samples through quantification of tau protein-derived peptides that are phosphorylated at various positions.

Specifically, depending on the type of the phosphorylated peptides, each stage of Alzheimer's disease may be determined and amyloid beta accumulation in the brain may be determined from a group of patients at the same cognitive function stage.

In one embodiment of the present invention, the early diagnosis of Alzheimer's disease may include early diagnosis of preclinical Alzheimer's disease and Alzheimer's mild cognitive impairment, and the classification of Alzheimer's disease stages may include a distinction between preclinical Alzheimer's disease stage, Alzheimer's mild cognitive impairment stage and Alzheimer's dementia stage.

Phosphorylation of tau protein, which is quantified for early diagnosis or stage classification of Alzheimer's disease, may include phosphorylation at Y394; S396; (T403/S404), Y394; S396, S396; S400; S404, S202, S198; S202, S199; S202, S404, S400; S404, T231 and/or T181 positions. Specifically, the tau protein-derived phosphorylated peptide for early diagnosis or stage classification of Alzheimer's disease may be phosphorylated peptides including phosphorylation at Y394; S396; (T403/S404), Y394; S396, S396; S400; S404, S202, S198; S202, S199; S202, S404, S400; S404, T231 and/or T181 positions in the tau protein represented by the amino acid sequence of SEQ ID NO: 14, wherein the phosphorylated peptide may consist of 3 to 50 amino acid sequences, specifically 5 to 30 amino acid sequences, and more specifically 9 to 21 amino acid sequences.

In one embodiment of the present invention, the early diagnosis of Alzheimer's disease may be an early diagnosis of preclinical Alzheimer's disease by distinguishing the patient from a normal control. A method for early diagnosis of preclinical Alzheimer's disease by distinguishing it from a normal control is to quantify tau protein-derived phosphorylated peptides including phosphorylation at Y394; S396; (T403/S404) positions, wherein, if the quantitative value is higher compared to the normal control having normal cognitive function without brain amyloid plaques or if the quantitative value is higher than a standard value set as a quantitative value of the control, it may be determined to be preclinical Alzheimer's disease. Specifically, when the quantified tau protein-derived phosphorylated peptide is at least 2-fold, preferably at least 3-fold, more preferably at least 4-fold, and even more preferably at least 5-fold compared to the normal control having normal cognitive function without brain amyloid plaques, it may be determined to be the preclinical Alzheimer's disease. Further, if it is higher than the standard value set as the quantitative value of the control, it may be determined to be the preclinical Alzheimer's disease.

In another embodiment, the early diagnosis of Alzheimer's disease may be an early diagnosis of Alzheimer's mild cognitive impairment by distinguishing it from a normal control. The method for early diagnosis of Alzheimer's mild cognitive impairment by distinguishing it from the normal control is to quantify tau protein-derived phosphorylated peptide including phosphorylation at one or more positions selected from the group consisting of Y394; S396, S396; S400; S404, S202, S198; S202, S199; S202, S404, S400; S404 and T231, and more preferably to quantify tau protein-derived phosphorylated peptide including phosphorylation corresponding to the group consisting of Y394; S396, S396; S400; S404, S202, S199; S202 and S404. As compared to the normal control having normal cognitive function without brain amyloid plaques, if the quantitative value is higher than that of the normal control or if the quantitative value is higher than the standard value set as the quantitative value of the control, it may be determined to be Alzheimer's mild cognitive impairment. Specifically, when the quantified tau protein-derived phosphorylated peptide is higher at least 2-fold, preferably at least 3-fold, more preferably at least 4-fold, and even more preferably at least 5-fold compared to the normal control having normal cognitive function without brain amyloid plaques, it may be determined to be Alzheimer's mild cognitive impairment. Further, if the phosphorylated peptide is higher than the standard value set as the quantitative value of the control, it may be determined to be Alzheimer's mild cognitive impairment.

In one embodiment of the present invention, the classification of Alzheimer's disease stages may include a distinction between a preclinical Alzheimer's disease stage and an Alzheimer's mild cognitive impairment stage. The method for classifying between the preclinical Alzheimer's disease stage and the Alzheimer's mild cognitive impairment stage is to quantify tau protein-derived phosphorylated peptide including phosphorylation at one or more positions selected from the group consisting of Y394; S396, S396; S400; S404, S202, S198; S202, S199; S202, S404, S400; S404 and T231, and preferably to quantify tau protein-derived phosphorylated peptide including phosphorylation corresponding to the group consisting of 394; S396, S202 and S404. If the quantitative value is higher as compared to a control of preclinical Alzheimer's disease or is higher than a standard value set as the quantitative value of the control, it may be determined to be the Alzheimer's mild cognitive impairment stage.

Specifically, if the quantified tau protein-derived phosphorylated peptide is higher 2-fold or more, preferably 3-fold or more, more preferably 4-fold or more, and even more preferably 5-fold or more compared to the control of preclinical Alzheimer's disease, it may be determined to be the Alzheimer's mild cognitive impairment stage. Further, if the phosphorylated peptide is higher than the standard value set as the quantitative value of the control, it may be determined to be the Alzheimer's mild cognitive impairment.

In another embodiment, the classification of Alzheimer's disease stages may include a distinction between an Alzheimer's mild cognitive impairment stage and an Alzheimer's dementia stage. The method for classifying between the Alzheimer's mild cognitive impairment stage and the Alzheimer's dementia stage is to quantify tau protein-derived phosphorylated peptide including phosphorylation at one or more positions selected from the group consisting of Y394; S396, S396; S400; S404, S202, S198; S202, S199; S202, S404, S400; S404, T231 and T181, more preferably to quantify tau protein-derived phosphorylated peptide including phosphorylation corresponding to the group consisting of S202, S404, T231 and T181. Further, if the quantitative value is higher compared to the control of Alzheimer's mild cognitive impairment or is higher than the standard value set as the quantitative value of the control, it may be determined to be the Alzheimer's dementia stage. Specifically, if the quantified tau protein-derived phosphorylated peptide is 2-fold or more, preferably 3-fold or more, more preferably 4-fold or more, and even more preferably 5-fold or more compared to the control of Alzheimer's mild cognitive impairment, it may be determined to be the Alzheimer's dementia stage. Further, if the phosphorylated peptide is higher than the standard value set as the quantitative value of the control, it may be determined to be the Alzheimer's dementia stage.

In the present invention, the biological sample is characterized in that it is one or more selected from the group consisting of whole blood, serum, plasma, cerebrospinal fluid, saliva, urine, sputum, lymph fluid and cells.

In the present invention, the tau protein-derived phosphorylated peptide may be obtained by converting proteins in the isolated biological sample into peptides using proteolytic enzymes. Specifically, the proteolytic enzymes may include one are more proteolytic enzymes selected from the group consisting of Lys-c, Arg-C, Asp-N, Gluc-C, Lys-N, thermolysin, elastase, Tryp-N, trypsin and chymotrypsin. In addition, the phosphorylated tau protein or tau protein fragment may be obtained by a phosphorylated protein or phosphorylated peptide enrichment process.

In the present invention, the quantification of the tau protein-derived phosphorylated peptide may be performed by mass spectrometry using the liquid chromatography-mass spectrometry (LC-MS) device. Specifically, the mass spectrometry method may be LC-SRM (Liquid Chromatography-Selected Reaction Monitoring), PRM (Parallel Reaction Monitoring), SIM (Selected Ion Monitoring), or MRM (Multiple Reaction Monitoring).

In the present invention, the quantified amount of phosphorylated peptide may be a ratio to a total amount of tau

The method of the present invention may further include the step of performing quantitative profiling on the quantified amount of phosphorylated peptide. Specifically, the inventive method may further include the step of performing quantitative profiling, characterized in that one or more of T test p-value analysis, ROC (Receiver-Operating Curve), and AUC (Area under the ROC curve) analysis are selected and executed on the quantified amount of phosphorylated peptide. The quantitative profiling is performed by injecting an isotope-substituted protein standard (stable isotope labeled protein standard) or a peptide standard (stable isotope labeled peptide standard) before or after producing peptides form proteins, and using an absolute quantitative value of phosphorylated peptide obtained through synthetic labeled peptides. The absolute quantification value may be compared as a ratio to a total amount of tau protein, wherein the amount of total tau protein may also be expressed an absolute quantitative value obtained from the isotope-substituted protein standard (stable isotope labeled protein standard) or the peptide standard (stable isotope labeled peptide standard). The relevant standard value can be implemented by setting an appropriate standard value according to specific embodiments. For example, by determining the threshold (cutoff) of the control range (upper limit in the case of increase/lower limit in the case of decrease), if the amount of phosphorylated tau protein fragment peptide fluctuates more than the threshold, it may be determined to be “significantly higher than the average.” Using an absolute content value of the quantified phosphorylated peptide and comparing the same with the absolute quantitative value corresponding to the control, if it is higher 2-fold or more, preferably 3-fold or more, more preferably 4-fold or more, and even more preferably 5-fold or more, it may be determined to be each corresponding stage.

In the present invention, early diagnosis of Alzheimer's disease may include identifying a subject for subsequent onset. Specifically, early diagnosis of preclinical Alzheimer's disease may further include identifying a subject about 5 to 25 years or less from the onset of Alzheimer's mild cognitive impairment, and early diagnosis of Alzheimer's mild cognitive impairment may further include identifying the subject about 5 to 25 years or less from the onset of Alzheimer's dementia.

Further, in the present invention, the classification of Alzheimer's disease stages may be performed in order to select only subjects corresponding to the Alzheimer's disease stage targeted by actual therapeutics in clinical trials for subjects, or to select subjects for administration of pharmaceutical compositions corresponding to each stage.

In another embodiment of the present invention, the determination of amyloid beta accumulation in the brain may be to determine amyloid beta accumulation in the brain in a normal cognitive function group, a patient group with mild cognitive impairment or a patient group with dementia.

Phosphorylation of tau protein quantified to determine amyloid beta accumulation in the brain may be phosphorylation at Y394; S396; (T403/S404), S396; S404, and/or S285; S289 positions. Specifically, the tau protein-derived phosphorylated peptide for determining amyloid beta accumulation in the brain may be phosphorylated peptides including phosphorylation at Y394; S396; (T403/S404), S396; S404, and/or S285; S289 positions in the tau protein represented by the amino acid sequence of SEQ ID NO: 14, and the phosphorylated peptide may be composed of 3 to 50 amino acid sequences including phosphorylation at the positions listed above, and specifically, may be composed of 5 to 30 amino acid sequences, and more specifically 9 to 21 amino acid sequences.

In one embodiment of the present invention, the determination of amyloid beta accumulation in the brain may be performed in a group with normal cognitive function. The determination of amyloid beta accumulation in the brain in the group with normal cognitive function may be performed by quantifying tau protein-derived phosphorylated peptides including phosphorylation at Y394; S396; (T403/S404) positions. If the quantitative value is higher compared to a control having normal cognitive function without amyloid beta accumulation in the brain, or is higher than the standard value set as the quantitative value of the control, it may be determined to be amyloid beta accumulation in the brain. Specifically, when the quantified tau protein-derived phosphorylated peptide is higher 2-fold or more, preferably 3-fold or more, more preferably 4-fold or more, and even more preferably 5-fold or more compared to a control having normal cognitive function without amyloid beta accumulation in the brain, it may be determined to be amyloid beta accumulation in the brain. Further, if the phosphorylated peptide is higher than the standard value set as the quantitative value of the control, it may be determined to be amyloid beta accumulation in the brain.

In another embodiment, the determination of amyloid beta accumulation in the brain may be performed in a group of patients with mild cognitive impairment. The determination of amyloid beta accumulation in the brain in the patient group with mild cognitive function may be performed by quantifying tau protein-derived phosphorylated peptides including phosphorylation at S396; S404 positions. If the quantitative value is lower compared to the mild cognitive impairment control without amyloid beta accumulation in the brain, or is lower than the standard value set as the quantitative value of the control, it may be determined to be amyloid beta accumulation in the brain. Specifically, when the quantified tau protein-derived phosphorylated peptide is lower 2-fold or more, preferably 3-fold or more, more preferably 4-fold or more, and even more preferably 5-fold or more compared to the mild cognitive impairment control without amyloid beta accumulation in the brain, it may be determined to be amyloid beta accumulation in the brain. Further, if the phosphorylated peptide is lower than the standard value set as the quantitative value of the control, it may be determined to be amyloid beta accumulation in the brain.

In another embodiment, the determination of amyloid beta accumulation in the brain may be performed in a group of dementia patients. The determination of amyloid beta accumulation in the brain in the dementia patient group may be performed by quantifying tau protein-derived phosphorylated peptides including phosphorylation at 285; S289 positions, and if the quantitative value is lower compared to the control of dementia patients without amyloid beta accumulation in the brain or if the quantitative value is lower than the standard value set as the quantitative value of the control, it may be determined to be amyloid beta accumulation in the brain. Specifically, when the quantified tau protein-derived phosphorylated peptide is lower 2-fold or more, preferably 3-fold or more, more preferably 4-fold or more, and even more preferably 5-fold or more compared to the dementia patient group without amyloid beta accumulation in the brain, it may be determined to be amyloid beta accumulation in the brain. Further, if the phosphorylated peptide is lower than the standard value set as the quantitative value of the control, it may be determined to be amyloid beta accumulation in the brain.

In the present invention, the determination of brain amyloid beta accumulation may be used for selecting only subjects corresponding to the brain amyloid beta accumulation targeted by actual therapeutics in clinical trials for the subjects, or for selecting subjects for administration of pharmaceutical compositions corresponding to brain amyloid beta accumulation in the brain at each stage. Further, the brain amyloid beta accumulation is used to diagnose diseases related to brain amyloid beta accumulation, or to determine whether an amyloid beta-related PET test is required. This may be for the purpose of determining whether or not there is brain amyloid accumulation wherein brain amyloid beta accumulation diseases may include Alzheimer's disease, Parkinson's disease, dementia, Lewy body dementia, Huntington's disease dementia, preclinical Alzheimer's disease, Down syndrome, or cognitive impairment.

In another aspect of the present invention, the present invention provides a composition for early diagnosis or stage classification of Alzheimer's disease, or for determination of amyloid beta accumulation in the brain, which includes a formulation capable of detecting phosphorylation sites at one or more positions selected from the group consisting of Y394; S396; (T403/S404), Y394; S396, S396; S400; S404, S202, S198; S202, S199; S202, S404, S400; S404, T231 and T181 in tau protein.

The phosphorylation site, the early diagnosis or stage classification of Alzheimer's disease, or the determination of amyloid beta accumulation in the brain would have the same meanings as described above.

In another aspect of the present invention, the present invention may provide a phosphorylated peptide for early diagnosis or stage classification of Alzheimer's disease, or for determination of amyloid beta accumulation in the brain. Specifically, the phosphorylated peptide may include phosphorylation at each position in the tau protein represented by the amino acid sequence of SEQ ID NO: 14, and may be composed of 3 to 50 amino acid sequences including phosphorylation at each position, may be composed of 5 to 30 amino acid sequences, and more specifically, may be composed of 9 to 21 amino acid sequences.

In one embodiment, it may be a tau protein-derived phosphorylated peptide including phosphorylation at Y394; S396; (T403/S404) positions for early diagnosis of preclinical Alzheimer's disease while distinguishing it from normal controls, and specifically, may be a phosphorylated peptide of SEQ ID NO: 4, but it is not limited thereto.

In another embodiment, it may be a tau protein-derived phosphorylated peptide including phosphorylation at Y394; S396, S396; S400; S404, S202, S198; S202, S199; S202, S404, S400; S404 and/or T231 positions for early diagnosis of Alzheimer's mild cognitive impairment by distinguishing it from normal controls, and preferably a tau protein-derived phosphorylated peptide including phosphorylation at Y394; S396, S396; S400; S404, S202, S199; S202 and/or S404 positions. Specifically, it may be the phosphorylated peptides of SEQ ID NO: 2, SEQ ID NO: 3, SEQ ID NO: 5, SEQ ID NO: 6, SEQ ID NO: 7, SEQ ID NO: 8, SEQ ID NO: 9 and/or SEQ ID NO: 12, and preferably the phosphorylated peptides of SEQ ID NO: 2, SEQ ID NO: 3, SEQ ID NO: 5, SEQ ID NO: 7 and/or SEQ ID NO: 8, but it is not limited thereto.

In another embodiment, it may be a tau protein-derived phosphorylated peptide including phosphorylation at Y394; S396, S396; S400; S404, S202, S198; S202, S199; S202, S404, S400; S404 and/or T231 positions in order to classify between the preclinical Alzheimer's disease stage and the Alzheimer's mild cognitive impairment stage, and preferably, a tau protein-derived phosphorylated peptide including phosphorylation at Y394; S396, S202 and/or S404 positions. Specifically, it may be the phosphorylated peptides of SEQ ID NO: 2, SEQ ID NO: 3, SEQ ID NO: 5, SEQ ID NO: 6, SEQ ID NO: 7, SEQ ID NO: 8, SEQ ID NO: 9 and/or SEQ ID NO: 12, and preferably the phosphorylated peptides of SEQ ID NO: 2, SEQ ID NO: 5 and/or SEQ ID NO: 8, but it is not limited thereto.

In another embodiment, it may be a tau protein-derived phosphorylated peptide including phosphorylation at Y394; S396, S396; S400; S404, S202, S198; S202, S199; S202, S404, S400; S404, T231 and/or T181 positions in order to classify between an Alzheimer's mild cognitive impairment stage and an Alzheimer's dementia stage, and preferably, a tau protein-derived phosphorylated peptide including phosphorylation at S202, S404, T231 and/or T181 positions. Specifically, it may be the phosphorylated peptides of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 12 and/or SEQ ID NO: 13, and preferably the phosphorylated peptides of SEQ ID NO: 5, SEQ ID NO: 8, SEQ ID NO: 12 and/or SEQ ID NO: 13, but it is not limited thereto.

In another embodiment, it may be a tau protein-derived phosphorylated peptide including phosphorylation at S394; S396; (T403/S404) positions for determination of amyloid beta accumulation in the brain in a group with normal cognitive function, specifically it may be the phosphorylated peptide of SEQ ID NO: 4, but it is not limited thereto.

In another embodiment, it may be a tau protein-derived phosphorylated peptide including phosphorylation at S396; S404 positions for determination of amyloid beta accumulation in the brain of a patient group with mild cognitive impairment, specifically, it may be the phosphorylated peptide of SEQ ID NO: 1, but it is not limited thereto.

In another embodiment, it may be a tau protein-derived phosphorylated peptide including phosphorylation at S285; S289 positions for determination of amyloid beta accumulation in the brain of a dementia patient group, specifically, it may be the phosphorylated peptide of SEQ ID NO: 11, but it is not limited thereto.

In the present invention, as described above, the hyperphosphorylation of tau protein is measured to enable early diagnosis and stage classification of Alzheimer's disease and determination of amyloid beta accumulation in the brain. Such hyperphosphorylation of tau protein is a main characteristic of tauopathy, thus it can be applied to not only Alzheimer's disease, but also to degenerative brain diseases related to tauopathy including progressive supranuclear palsy (PSP), corticobasal degeneration (CBD), Pick's disease, frontotemporal dementias with Parkinsonism liked to chromosome 17 (FTDP-17), chronic traumatic encephalopathy (CTE).

Hereinafter, the present invention will be described in more detail through examples. However, these examples are for illustrative purposes only and the scope of the present application is not limited to these examples.

Diagnosing Alzheimer's disease through blood has advantages in many aspects such as non-invasiveness, costs, and facilities, but since blood exchanges substances with various tissues, including the brain, the specificity of the diagnosis through blood indicators is often decreased. Further, in existing cases using the immunoassay method, there were limitations due to the availability, specificity, sensitivity, and the like of antibodies.

Accordingly, various pretreatment methods are used to solve problems caused by the characteristics of blood samples that interfere with blood sample analysis, such as an immunodepletion method to remove a small number of high-concentration proteins, a process of diluting high-concentration protein concentration and concentrating low-concentration phosphorylated protein using bead various peptides which is a method to reduce the dynamic range of protein concentration, and fractionation to reduce complexity, and the like were attempted, but each entailed limitations.

Despite many studies to overcome these characteristics of blood samples, the analysis of blood phosphorylated proteins suffers from significantly lower phosphorylated peptide enrichment efficiency and selectivity compared to tissue or cell samples.

Accordingly, the present invention sought to optimize the phosphorylated peptide enrichment process for blood samples without depletion while having high complexity and wide dynamic range, and to develop a method with the highest phosphorylated peptide enrichment efficiency. Therefore, even though the same amount of plasma sample was used, it was found that the number of identified phosphorylated peptides, phosphorylated proteins, phosphorylated sites, and spectra corresponding to the phosphorylated peptides varied greatly depending on the type of phosphorylated peptide enrichment process or buffer composition. The method of the present invention may use a concentration process with high phosphorylated peptide enrichment efficiency, but it is not limited thereto.

### Example 1: Quantification of Tau Protein-Derived Phosphorylated Peptide

For early diagnosis of Alzheimer's disease, it was attempted to quantify tau protein-derived phosphorylated peptides and determine the preclinical Alzheimer's disease (preclinical AD) stage or mild cognitive impairment of Alzheimer's disease (MCI-AD) based on the quantification results.

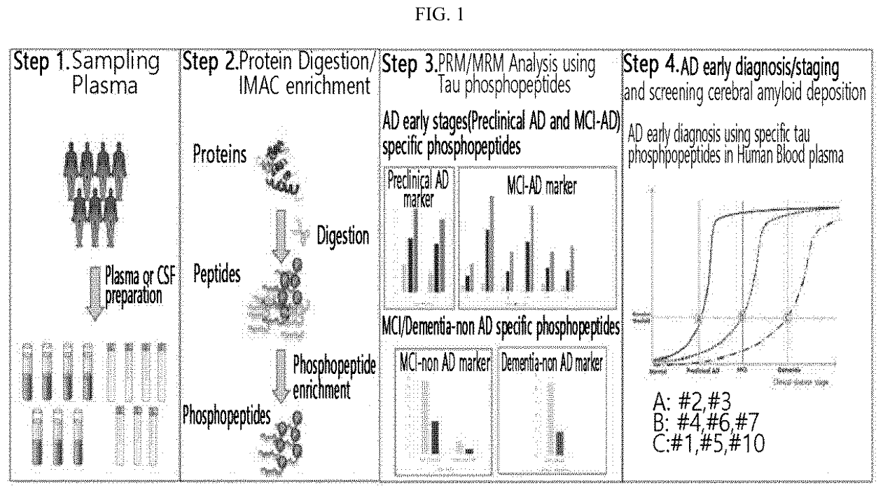

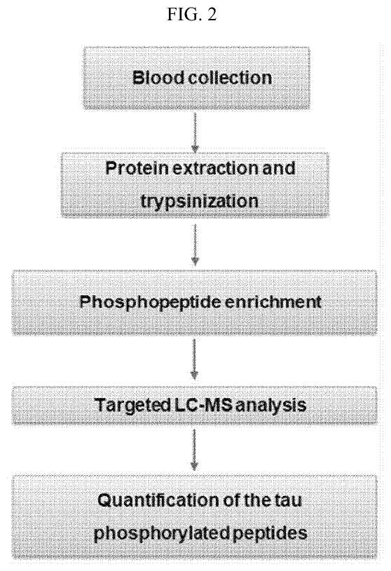

A schematic diagram of the overall experiment is shown in FIG. 1, and the specific experimental method is as shown in FIG. 2. Specifically, the plasma for each group used in the experiment was obtained from the same amount of blood samples obtained and combined from 10 patients in each group.

Herein, each group refers to:

Normal−: A group with normal cognitive function and no amyloid deposition.

Normal+ (preclinical AD): A group with normal cognitive function but amyloid deposition found.

MCI−: A group with mild cognitive impairment but without amyloid deposition.

MCI+ (MCI-AD): A group with mild cognitive impairment and amyloid deposition found.

AD−: A group that has experienced a decline in cognitive function sufficient to be diagnosed as dementia but has not developed amyloid deposition.

AD+ (Dementia-AD): A group diagnosed with Alzheimer's dementia due to a decline in cognitive function sufficient to be diagnosed as dementia and amyloid deposition discovered.

Plasma samples were lysed with lysis buffer (50 mM Tris HCl pH 8.0, 8 M urea, 25× Complete (protease inhibitor), 10× phosphatase inhibitor). The dissolved sample was subjected to a disulfide reduction reaction at 37° C. by adding DTT (final concentration 20 mM) for 30 minutes to 1 hour at room temperature, and then adding iodoacetamide (final concentration 50 mM) and conducting sulfhydryl group alkylation reaction for 30 minutes at room temperature under dark condition. The treated sample was diluted with 50 mM NH₄HCO₃ so that the final concentration of urea was 2 M or less, trypsinized at a ratio of trypsin to sample of 1:50, and reacted at 37° C. for 18 hours. Afterwards, it was desalted using C-18 reversed-phase to obtain a peptide mixture. Immobilized metal ion affinity chromatography (IMAC) was performed using a Fe-IMAC column adsorbed with iron ions (Fe³⁺) as a ligand. The obtained peptide mixture was dissolved in a loading buffer solution, and then injected into a Fe-IMAC column equilibrated with the same loading buffer solution. The Fe-IMAC column loaded with the sample was washed with a loading buffer solution, and then an eluate in which phosphorylated peptides were concentrated by the elution buffer solution was obtained. The obtained eluate was quantified through mass spectrometry using liquid chromatography-parallel reaction monitoring (LC-PRM).

The list of phosphorylated peptides quantified herein is as shown in Table 1 below. Specifically, the numbered phosphorylation sites are described from the entire tau protein amino acid sequence (SEQ ID NO: 14), and the phosphorylation site of each peptide was underlined and bolded.

TABLE 1

| Serial | | Phosphorylation | |
|---|---|---|---|
| No. | Amino acid sequence | site | Sequence list |
| #1 | TDHGAEIVYK S PVVSGDT S PR | S396; S404 | SEQ ID NO: 1 |
| #2 | TDHGAEIV Y K S PVVSGDTSPR | Y394; S396 | SEQ ID NO: 2 |
| #3 | TDHGAEIVYK S PVV S GDT S PR | S396; S400; S404 | SEQ ID NO: 3 |
| #4 | TDHGAEIV Y K S PVVSGD TS PR | Y394; S396; | SEQ ID NO: 4 |
| | | (T403/S404) | |
| #5 | SGYSSPG S PGTPGSR | S202 | SEQ ID NO: 5 |
| #6 | SGY S SPG S PGTPGSR | S198; S202 | SEQ ID NO: 6 |
| #7 | SGYS S PG S PGTPGSR | S199; S202 | SEQ ID NO: 7 |
| #8 | SPVVSGDT S PR | S404 | SEQ ID NO: 8 |
| #9 | SPVV S GDT S PR | S400; S404 | SEQ ID NO: 9 |
| #10 | KLDLSNVQ S K | S289; N286(deami) | SEQ ID NO: 10 |
| #11 | KLDL S NVQ S K | S285; S289 | SEQ ID NO: 11 |
| #12 | VAVVR T PPK | T231 | SEQ ID NO: 12 |
| #13 | TPPAPK T PPSSGEPPK | T181 | SEQ ID NO: 13 |

### Example 2: Confirmation of Quantitative Results of Tau Protein-Derived Phosphorylated Peptide

The results of quantifying phosphorylated peptides for each group through the method of Example 1 above are shown.

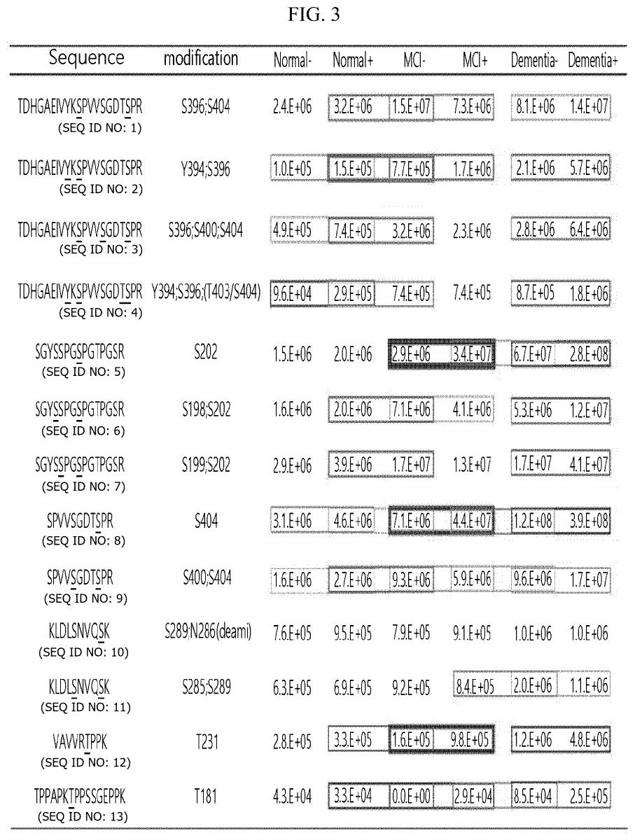

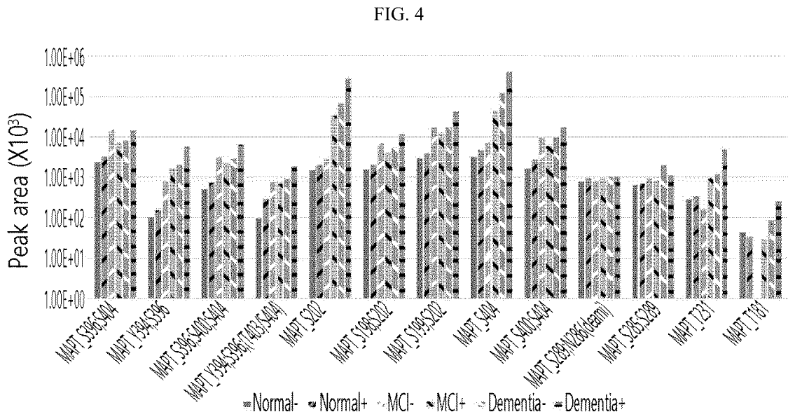

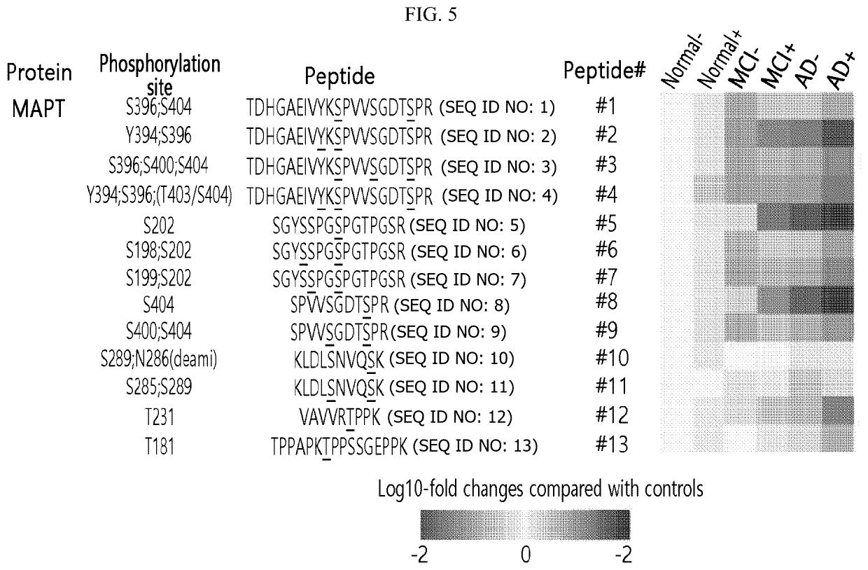

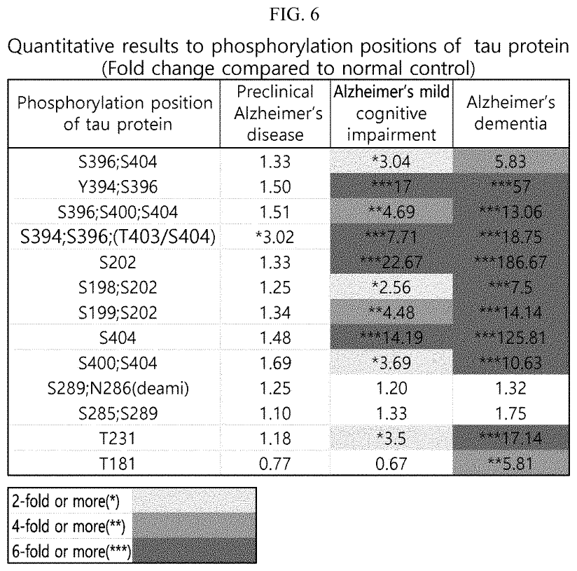

Specifically, in FIG. 3, the quantification of phosphorylated peptides was described using numerical values, and this was schematized using a histogram (FIG. 4) and a heatmap (FIG. 5). The heatmap plotted with the fold change compared to the normal control, and the values are shown in FIG. 6.

As shown in FIGS. 3 to 6, it was confirmed that the phosphorylated peptides derived from tau proteins were specifically increased in Alzheimer's disease, and some of these phosphorylated peptides were found in the early stages of Alzheimer's dementia, i.e. preclinical AD and MCI-AD that the number has already more than doubled.

### Example 3: Quantitative Analysis of Tau Protein-Derived Phosphorylated Peptides

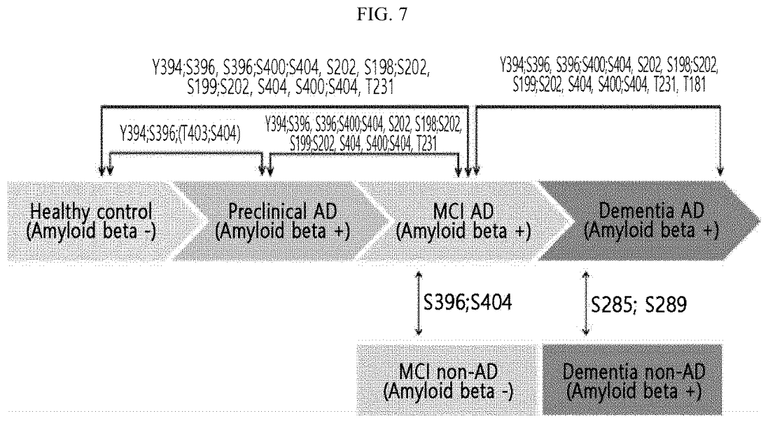

Through the results of Example 2, it was confirmed that some phosphorylated peptides had already increased in the early Alzheimer's dementia stage. As shown in FIG. 7, there was a specific increase according to each stage, and a specific change in amyloid accumulation in the brain at each stage was confirmed in more detail.

### 3-1: Phosphorylated Peptide Capable of Early Diagnosis in Preclinical AD Stages

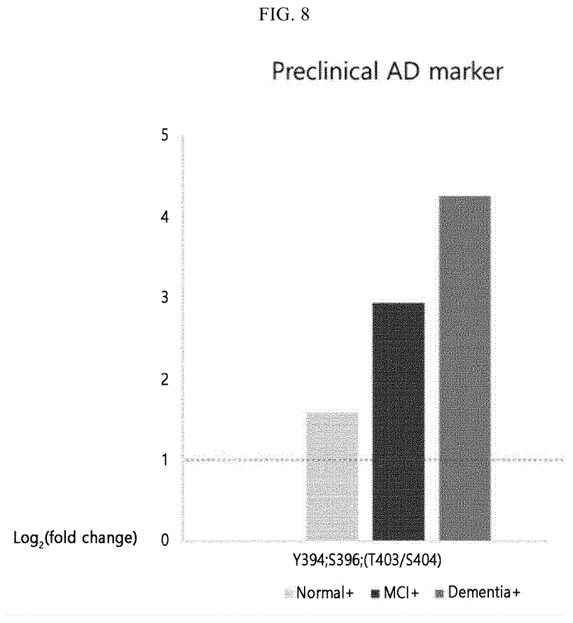

FIG. 8 illustrates results of quantifying #4 phosphorylated peptide. Compared to the Normal-group, it was confirmed that the phosphorylated peptide measurement values of the Normal+ group, that is, the group with normal cognitive function but diagnosed as preclinical AD due to the discovery of amyloid deposition, were increased more than 2-fold.

From this result, it was confirmed that through quantification of #4 phosphorylated peptide, subjects in the preclinical AD stage can be diagnosed even if symptoms do not yet appear.

### 3-2: Phosphorylated Peptide Capable of Early Diagnosis at the MCI-AD Stage

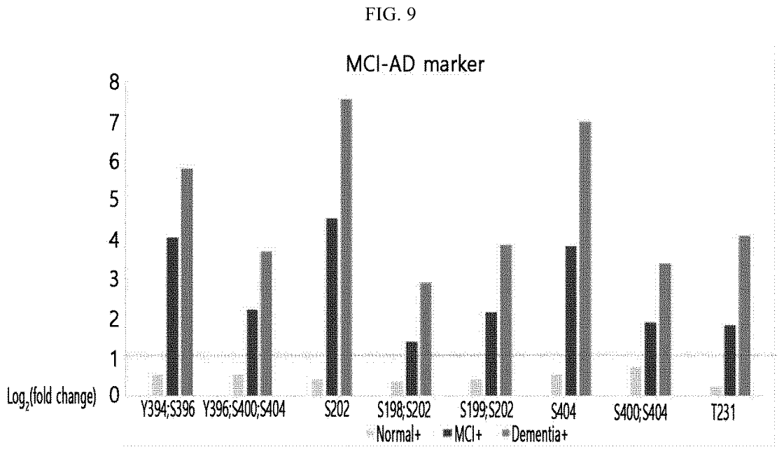

FIG. 9 illustrates results of quantifying phosphorylated peptides #2, #3, #5, #6, #7, #8, #9 and #12. Although no significant increase was observed in the Normal+ group, it was confirmed that the phosphorylated peptide measurement values of the MCI+ group, that is, the group with mild cognitive impairment and in which amyloid deposition was found, were increased more than 2-fold.

From this result, it was confirmed that, through the quantification of #2, #3, #5, #6, #7, #8, #9 and #12 phosphorylated peptides, subjects who not only have mild cognitive impairment but are also likely to develop dementia in the future, can be diagnosed.

3-3: Phosphorylated Peptide that can Distinguish Amyloid Deposition in the Same Stage of MCI

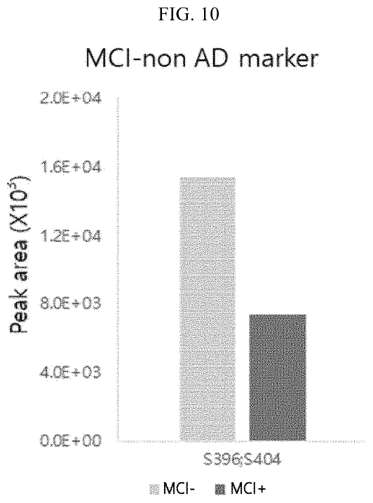

FIG. 10 illustrates results of quantifying #1 phosphorylated peptide. It was confirmed that the measured values increased in the MCI− group compared to the MCI+ group.

These results indicate that, even in subjects at the same stage of mild cognitive impairment, amyloid deposition did not occur and thus could be used to classify whether or not the subject has non-Alzheimer's mild cognitive impairment.

3-4: Phosphorylated Peptide that can Distinguish Amyloid Deposition at the Same Stage of AD

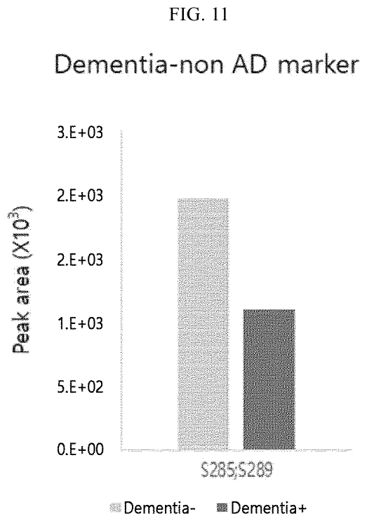

FIG. 11 illustrates results of quantifying #11 phosphorylated peptide. It was confirmed that the measured values were increased in the AD− group compared to the AD+ group.

These results indicate that even in subjects at the same stage of dementia, amyloid deposition did not occur and can be used to classify whether or not the subject has non-Alzheimer's dementia.

### Example 4: Method for Selecting Patient Groups or Normal Groups in Separate Stages of Alzheimer's Disease for Non-Clinical/Clinical Trials by Quantifying Phosphorylated Peptides Derived from Dementia-Related Proteins, Including Tau, in the Blood

According to the above-described results, it is possible to provide a method to increase the probability of clinical trial success through quantification of phosphorylated peptides derived from dementia-related proteins, including tau, in the blood in order to select Alzheimer's disease groups or normal groups. In clinical trials to prove drug efficacy, results are determined by showing statistical significance to determine whether the expected effects predicted in advance are achieved for clinical trial participants. As shown in FIG. 7, when classifying stages of Alzheimer's disease through the quantification of phosphorylated peptides derived from dementia-related proteins including tau used in the present invention, Alzheimer's disease patients exactly targeted by the new drug are included as test subjects so that the probability of clinical trial success can be increased as much as possible. In the case of Alzheimer's disease, whether or not symptoms are improved due to drug efficacy is measured by biomarkers using images/cerebrospinal fluid or questionnaires. However, biomarkers using images/cerebrospinal fluid have problems such as costs, facilities, accessibility to sample collection, side effects and the like. Further, in the case of the questionnaire method or questionnaire, there are problems in that the scale is not detailed and objective and standardized quantification is difficult.

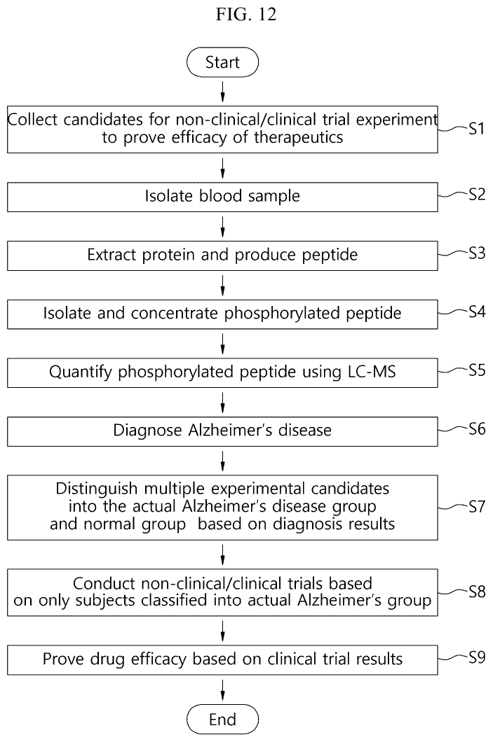

In order to solve this problem, the present invention sought to increase the probability of clinical trial success as much as possible by selecting only Alzheimer's disease patients exactly targeted by the treatment as clinical trial subjects for efficacy evaluation. In the case of central nervous system drugs, because of high response rate to placebo, reducing the heterogeneity of a target group and establishing biomarkers capable of predicting drug response would be important strategies in increasing the success rate. In particular, in the case of Alzheimer's disease, due to difficulty in diagnosis itself, it is more important to select the exact subjects targeted by the treatment and to select the drug response group. Therefore, the present invention aims to increase the probability of clinical trials using the method shown in FIG. 12 in order to quantify the phosphorylated peptides derived from dementia-related proteins, including tau, in the selection of subjects or target models.

Referring to FIG. 12, a first step (S1) of recruiting candidates for clinical trials in order to prove the efficacy of a therapeutic for Alzheimer's disease is performed. Thereafter, for a plurality of experimental candidates, the steps of: preparing blood samples of the experimental candidates (S2); converting proteins in the blood into peptides using proteolytic enzymes (S3); concentrating the phosphorylated peptides derived from dementia-related proteins including tau by applying the concentration process of phosphorylated peptides to the obtained peptides (S4); quantifying the concentrated tau protein-derived phosphorylated peptide through mass spectrometry using a liquid chromatography-mass spectrometry (LC-MS) device (S5); and diagnosing whether Alzheimer's disease exists or not based on the quantitative results (S6) are performed. After the diagnosis result is obtained through step S6, a step (S7) of distinguishing the plurality of experimental candidates into a patient group corresponding to the Alzheimer's disease group targeted by the treatment and a normal patient group may be performed based on the diagnosis results. At this time, through a step (S8) of conducting clinical trials based on only the patient group corresponding to the actual target stage of Alzheimer's disease and a step (S9) of proving the effectiveness of the treatment based on the clinical trial results, only the group corresponding to the Alzheimer's disease stage exactly targeted by new therapeutics are included as clinical trial subjects, thereby increasing the probability of clinical trial success as much as possible.

From the above description, those skilled in the art to which the present invention pertains will understand that the present invention can be implemented in other specific forms without changing its technical idea or essential features. In this regard, the embodiments described above should be understood to be illustrative and not restrictive in all respects. The scope of the present invention should be construed as including the meaning and scope of the claims described below rather than the detailed description above, and further include all changes or modified forms derived from the equivalent concepts thereof.

1. A method for early diagnosis and/or stage classification of Alzheimer's disease, the method comprising:
(a) obtaining a tau protein-derived phosphorylated peptide from an isolated biological sample; and
(b) quantifying the tau protein-derived phosphorylated peptide.

2. The method according to claim 1, wherein the early diagnosis of Alzheimer's disease includes early diagnosis of preclinical Alzheimer's disease and Alzheimer's mild cognitive impairment; and
the stage classification of Alzheimer's disease includes a distinction between a preclinical Alzheimer's disease stage, an Alzheimer's mild cognitive impairment stage, and an Alzheimer's dementia stage.

3. The method according to claim 1, wherein the the tau protein-derived phosphorylated peptide is a phosphorylated peptide including phosphorylation at one or more positions selected from the group consisting of Y394; S396; (T403/S404), Y394; S396, S396; S400; S404, S202, S198; S202, S199; S202, S404, S400; S404, T231 and T181.

4. The method according to claim 1, wherein the tau protein-derived phosphorylated peptide consists of 5 to 30 amino acid sequences including phosphorylation at one or more positions selected from the group consisting of Y394; S396; (T403/S404), Y394; S396, S396; S400; S404, S202, S198; S202, S199; S202, S404, S400; S404, T231 and T181 in the tau protein represented by the amino acid sequence of SEQ ID NO: 14.

5. The method according to claim 1, further comprising (c) comparing the quantified tau protein-derived phosphorylated peptide with a control.

6. The method according to claim 1, wherein the early diagnosis of Alzheimer's disease is an early diagnosis of the preclinical Alzheimer's disease by distinguishing it from a normal control, and involves quantifying a tau protein-derived phosphorylated peptide including phosphorylation at Y394; S396; (T403/S404) positions, and
wherein, if the quantitative value is higher compared to a normal control having normal cognitive function without brain amyloid plaques or higher than a standard value set as a quantitative value of the control, it is determined to be the preclinical Alzheimer's disease.

7. The method according to claim 1, wherein the early diagnosis of Alzheimer's disease is an early diagnosis of Alzheimer's mild cognitive impairment by distinguishing it from a normal control, and involves quantifying a tau protein-derived phosphorylated peptide including phosphorylation at one or more positions selected from the group consisting of Y394; S396, S396; S400; S404, S202, S198; S202, S199; S202, S404, S400; S404 and T231, and
wherein, if the quantitative value is higher compared to a normal control having normal cognitive function without brain amyloid plaques or higher than a standard value set as a quantitative value of the control, it is determined to be the Alzheimer's mild cognitive impairment.

8. The method according to claim 1, wherein the stage classification of Alzheimer's disease includes a distinction between a preclinical Alzheimer's disease stage and an Alzheimer's mild cognitive impairment stage, and involves quantifying a tau protein-derived phosphorylated peptide including phosphorylation at one or more positions selected from the group consisting of Y394; S396, S396; S400; S404, S202, S198; S202, S199; S202, S404, S400; S404 and T231, and
wherein, if the quantitative value is higher compared to a control of preclinical Alzheimer's disease or is higher than a standard value set as a quantitative value of the control, it is determined to be the Alzheimer's mild cognitive impairment stage.

9. The method according to claim 1, wherein the stage classification of Alzheimer's disease includes a distinction between an Alzheimer's mild cognitive impairment stage and an Alzheimer's dementia stage, and involves quantifying a tau protein-derived phosphorylated peptide including phosphorylation at one or more positions selected from the group consisting of Y394; S396, S396; S400; S404, S202, S198; S202, S199; S202, S404, S400; S404, T231 and T181, and
wherein, if the quantitative value is higher compared to a control of Alzheimer's mild cognitive impairment or is higher than a standard value set as a quantitative value of the control, it is determined to be the Alzheimer's dementia stage.

10. The method according to claim 1, wherein, in the step (a), the tau protein-derived phosphorylated peptide is obtained by converting proteins in the isolated biological sample into peptides using proteolytic enzymes.

11. The method according to claim 10, wherein the proteolytic enzyme is one or more proteolytic enzymes selected from the group consisting of Lys-c, Arg-C, Asp-N, Gluc-C, Lys-N, thermolysin, elastase, Tryp-N, trypsin and chymotrypsin.

12. The method according to claim 10, further comprising concentrating the obtained tau protein-derived phosphorylated peptide by a phosphorylated peptide enrichment process.

13. The method according to claim 1, wherein the biological sample is one or more selected from the group consisting of whole blood, serum, plasma, cerebrospinal fluid, saliva, urine, sputum, lymph fluid and cells.

14. The method according to claim 1, wherein, in the step (b), the quantification of the tau protein-derived phosphorylated peptide is performed by mass spectrometry using a liquid chromatography-mass spectrometry (LC-MS) device.

15. The method according to claim 14, wherein the mass spectrometry is LC-SRM (Liquid Chromatography-Selected Reaction Monitoring), PRM (Parallel Reaction Monitoring), SIM (Selected Ion Monitoring), or MRM (Multiple Reaction Monitoring).

16. The method according to claim 1, further comprising performing quantitative profiling, characterized in that one or more of T test p-value analysis, ROC (Receiver-Operating Curve), and AUC (Area under the ROC curve) analysis are selected and executed on the quantified amount of phosphorylated peptide.

17. A method for determination of amyloid beta accumulation in the brain, the method comprising:
(a) obtaining a tau protein-derived phosphorylated peptide from an isolated biological sample; and
(b) quantifying the tau protein-derived phosphorylated peptide.

18. The method according to claim 17, wherein the determination of amyloid beta accumulation in the brain is to determine whether amyloid beta accumulation is present in the brain in a normal cognitive function group, a patient group with mild cognitive impairment or a patient group with dementia.

19. The method according to claim 17, wherein the tau protein-derived phosphorylated peptide is a phosphorylated peptide including phosphorylation at one or more positions selected from the group consisting of Y394; S396; (T403/S404), S396; S404 and S285; S289.

20. The method according to claim 17, wherein the tau protein-derived phosphorylated peptide consists of 5 to 30 amino acid sequences including phosphorylation at one or more positions selected from the group consisting of Y394; S396; (T403/S404), S396; S404 and S285; S289 in the tau protein represented by the amino acid sequence of SEQ ID NO: 14.

21. The method according to claim 17, further comprising (c) comparing the quantified tau protein-derived phosphorylated peptide with a control.

22. The method according to claim 17, wherein the determination of amyloid beta accumulation in the brain is performed in a normal group with normal cognitive function, and involves quantifying a tau protein-derived phosphorylated peptide including phosphorylation at Y394; S396; (T403/S404) positions, and
wherein, if the quantitative value is higher compared to a control having normal cognitive function without amyloid beta accumulation in the brain or is higher than a standard value set as a quantitative value of the control, it is determined to be amyloid beta accumulation in the brain.

23. The method according to claim 17, wherein the determination of amyloid beta accumulation in the brain is performed in a patient group having mild cognitive impairment, and involves quantifying a tau protein-derived phosphorylated peptide including phosphorylation at S396; S404 positions, and
wherein, if the quantitative value is lower compared to a control of patients having mild cognitive impairment without amyloid beta accumulation in the brain or is lower than a standard value set as a quantitative value of the control, it is determined to be amyloid beta accumulation in the brain.

24. The method according to claim 17, wherein the determination of amyloid beta accumulation in the brain is performed in a dementia patient group, and involves quantifying a tau protein-derived phosphorylated peptide including phosphorylation at S285; S289 positions, and
wherein, if the quantitative value is lower compared to a control of dementia patients without amyloid beta accumulation in the brain or is lower than a standard value set as a quantitative value of the control, it is determined to be amyloid beta accumulation in the brain.

25. A composition for early diagnosis or stage classification of Alzheimer's disease, or for determination of amyloid beta accumulation in the brain, comprising:
a formulation adapted to detect a phosphorylation site at one or more positions selected from the group consisting of Y394; S396; (T403/S404), Y394; S396, S396; S400; S404, S202, S198; S202, S199; S202, S404, S400; S404, T231 and T181 in tau protein.


SEQUENCE LISTING (14 sequences)

SEQ ID NO: 1 - protein, 21 aa, synthetic construct
  REGION 1..21: note = phophorylated peptide
  SITE 11: note = MISC_FEATURE - PHOSPHORYLATION
  SITE 19: note = MISC_FEATURE - PHOSPHORYLATION
TDHGAEIVYKSPVVSGDTSPR

SEQ ID NO: 2 - protein, 21 aa, synthetic construct
  REGION 1..21: note = phophorylated peptide
  SITE 9: note = MISC_FEATURE - PHOSPHORYLATION
  SITE 11: note = MISC_FEATURE - PHOSPHORYLATION
TDHGAEIVYKSPVVSGDTSPR

SEQ ID NO: 3 - protein, 21 aa, synthetic construct
  REGION 1..21: note = phophorylated peptide
  SITE 11: note = MISC_FEATURE - PHOSPHORYLATION
  SITE 15: note = MISC_FEATURE - PHOSPHORYLATION
  SITE 19: note = MISC_FEATURE - PHOSPHORYLATION
TDHGAEIVYKSPVVSGDTSPR

SEQ ID NO: 4 - protein, 21 aa, synthetic construct
  REGION 1..21: note = phophorylated peptide
  SITE 9: note = MISC_FEATURE - PHOSPHORYLATION
  SITE 11: note = MISC_FEATURE - PHOSPHORYLATION
  REGION 18..19: note = MISC_FEATURE - PHOSPHORYLATION
TDHGAEIVYKSPVVSGDTSPR

SEQ ID NO: 5 - protein, 15 aa, synthetic construct
  REGION 1..15: note = phophorylated peptide
  SITE 8: note = MISC_FEATURE - PHOSPHORYLATION
SGYSSPGSPGTPGSR

SEQ ID NO: 6 - protein, 15 aa, synthetic construct
  REGION 1..15: note = phophorylated peptide
  SITE 4: note = MISC_FEATURE - PHOSPHORYLATION
  SITE 8: note = MISC_FEATURE - PHOSPHORYLATION
SGYSSPGSPGTPGSR

SEQ ID NO: 7 - protein, 15 aa, synthetic construct
  REGION 1..15: note = phophorylated peptide
  SITE 5: note = MISC_FEATURE - PHOSPHORYLATION
  SITE 8: note = MISC_FEATURE - PHOSPHORYLATION
SGYSSPGSPGTPGSR

SEQ ID NO: 8 - protein, 11 aa, synthetic construct
  REGION 1..11: note = phophorylated peptide
  SITE 9: note = MISC_FEATURE - PHOSPHORYLATION
SPVVSGDTSPR

SEQ ID NO: 9 - protein, 11 aa, synthetic construct
  REGION 1..11: note = phophorylated peptide
  SITE 5: note = MISC_FEATURE - PHOSPHORYLATION
  SITE 9: note = MISC_FEATURE - PHOSPHORYLATION
SPVVSGDTSPR

SEQ ID NO: 10 - protein, 10 aa, synthetic construct
  REGION 1..10: note = phophorylated peptide
  SITE 9: note = MISC_FEATURE - PHOSPHORYLATION
  SITE 6: note = MISC_FEATURE - deamination
KLDLSNVQSK

SEQ ID NO: 11 - protein, 10 aa, synthetic construct
  REGION 1..10: note = phophorylated peptide
  SITE 5: note = MISC_FEATURE - PHOSPHORYLATION
  SITE 9: note = MISC_FEATURE - PHOSPHORYLATION
KLDLSNVQSK

SEQ ID NO: 12 - protein, 9 aa, synthetic construct
  REGION 1..9: note = phophorylated peptide
  SITE 6: note = MISC_FEATURE - PHOSPHORYLATION
VAVVRTPPK

SEQ ID NO: 13 - protein, 16 aa, synthetic construct
  REGION 1..16: note = phophorylated peptide
  SITE 7: note = MISC_FEATURE - PHOSPHORYLATION
TPPAPKTPPSSGEPPK

SEQ ID NO: 14 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL